(19) United States
(12) Patent Application Publication
(10) Pub. No.: US 2013/0253255 A1
(43) Pub. Date: Sep. 26, 2013

(54) Brachytherapy Seed, Methodology and Calculating Dose of Brachytherapy and Method of Treatment

(71) Applicant: Wayne Van Niekerk, Benoni (ZA)

(72) Inventor: Wayne Van Niekerk, Benoni (ZA)

(73) Assignee: NORTHERN ONCOLOGY (PTY) LTD, Benoni (ZA)

(21) Appl. No.: 13/885,855

(22) PCT Filed: Nov. 17, 2011
(86) PCT No.: PCT/IB11/55151

§ 371 Date: May 16, 2013

(30) Foreign Application Priority Data
Nov. 18, 2010 (ZA) 2010/08253

(51) Int. Cl. A61N 5/10; B65B 7/28

(52) U.S. Cl. CPC A61N 5/1027; B65B 7/28
USPC 600/8; 53/467

(57) ABSTRACT

The invention provides for a brachy-therapy seed, wherein the seed includes at least two disparate radionuclides of a chemical element, the radionuclides encapsulated to form a brachytherapy seed and wherein the combination of the disparate radionuclides of the chemical element is used to augment dosimetric parameters and radiobiological characteristics of the brachytherapy seed. The invention also provides a method of manufacturing a brachytherapy seed that includes the steps of bombarding elemental silver with high energy protons produced by a cyclotron to obtain a target of 100 Pd and 103 Pd; dissolving and passing the target through a column of resin beads; placing at least one resin bead having adsorbed 100 Pd and 103 Pd into a titanium tube that is closed at one end; inserting a radiopaque member into the titanium tube; and closing the opposite end of the titanium tube.

### FIELD OF THE INVENTION

The invention is in the field of internal radiotherapy, sealed source radiotherapy, curietherapy or endocurietherapy, as a form of radiotherapy, where a radionuclide is encapsulated to form what is commonly known as a brachytherapy seed.

### BACKGROUND

The inventors are aware of the use of internal radiotherapy, sealed source radiotherapy, curietherapy or endocurietherapy, as a form of radiotherapy, where a radionuclide is encapsulated (brachytherapy seed) and is placed inside or next to an anatomical area requiring treatment. Brachytherapy is commonly used as a treatment for cancers such as cervical, prostate, breast and skin cancer.

The inventors are also aware that current radionuclides used in brachytherapy are often not the optimal radionuclide, due to cost considerations. In this respect ¹²⁵I (59 days half-life) is often used but it is known that the use of alternative (more expensive) radionuclides may result in tumour control at a much lower dose and lower mortality.

The inventor believes that a need exists for a brachytherapy seed that optimizes treatment and is more cost effective than currently available brachytherapy seeds.

### SUMMARY OF THE INVENTION

Definitions for the purpose of interpreting this specification:

Dosimetry—the process or method of measuring the dosage of ionizing radiation.

Brachytherapy—a form of radiotherapy where a radiation source (a brachytherapy seed) is placed inside or next to the area requiring treatment.

According to an aspect of the invention, there is provided a brachytherapy seed, wherein the seed includes at least two disparate radionuclides of a chemical element, the radionuclides encapsulated to form a brachytherapy seed and wherein the combination of the disparate radionuclides of the chemical element is used to augment dosimetric parameters and radiobiological characteristics of the brachytherapy seed.

The half-lives of the disparate radionuclides may be different and may result in different energies and decay properties. The dosimetric characteristics of the disparate radionuclides may thus be different, and the final dose distribution and radiobiological effectiveness of the resultant radiation may thus be dependent on the combination of the dosimetric characteristics of the combination of the type of radionuclides used in the brachytherapy seed.

The ratio of the disparate radionuclides, and therefore the dosimetry of the disparate radionuclides, may differ from the time of calibration to time of use.

A ratio at the time of use may be derived from a ratio at time of calibration.

The ratio at time of use may be derived from the ratio at time of calibration by using the published half-lives of the radionuclides and the decay equation A=A₀e^{-In(2).t/t1/2}

The dosimetric parameters may be determined by using mathematical modelling, wherein the disparate radionuclides may be considered separately and the theoretic dose distributions may be calculated and converted into desired parameters.

The mathematical modelling may be the Monte Carlo simulation wherein the disparate radionuclides may be considered separately and the theoretic dose distributions may be calculated and converted into the TG43 parameters¹. ¹ AAPM Radiation Therapy Task Group No. 43 (Med Phys 22(2) February 1995: 209-234 updated Med Phys 31 (3) March 2004: 633-674)

The dosimetric parameters may be determined by measuring the dosimetry around an actual seed at two or more different times, the times being long enough to show material changes in the dose distribution.

The dosimetric parameters may be determined by using a linear quadratic model to determine the relative biological effective (RBE) of the dosimetry before combining the radionuclides.

Utilising the known changes in the ratios and the measured changes in the dose distribution may assist in determining the dosimetric contributions of the radionuclides.

The radionuclides may be Palladium (Pd).

The radionuclides may include ¹⁰⁰Pd and ¹⁰³Pd radionuclides.

¹⁰⁰Pd and ¹⁰³Pd radionuclides may be obtained by bombarding a natural silver target with high energy protons produced by a cyclotron.

It is to be appreciated from this specification the radionuclides may be obtained by any suitable means, including but not limited to nuclear reactors, particle accelerators and/or radionuclide generators.

The brachytherapy seed may, at the time of manufacture, include a range of 5-25% ¹⁰⁰Pd.

The brachytherapy seed may, at the time of manufacture, typically include 16% ¹⁰⁰Pd.

Treatment planning software may facilitate the percentage of the ¹⁰⁰Pd at the time of implantation relative a malignancy in a patient and the physical half-lives of the ¹⁰⁰Pd and the ¹⁰³Pd.

The dosimetry and half-life of each radionuclide may be calculated separately.

The ¹⁰⁰Pd dosimetry may be weighted so that the doses of the ¹⁰⁰Pb is brought into line with the RBE of ¹⁰³Pd before the finalisation of the dosimetry.

Weighting the ¹⁰⁰Pd dosimetry may have the desired effect of resulting in a dosimetric equivalent of a pure ¹⁰³Pd seed.

The brachytherapy seed may include a column of resin beads.

The radionuclides may absorb onto the resin. Typically, more than 95% of the total Pd activity may absorb onto the resin.

The brachytherapy seeds may include a radiopaque substance for radiographic visualization of the brachytherapy bead.

The radiopaque substance may include any suitable material and may typically be in the form of a lead or gold bead.

The radiopaque beads may be sandwiched between the resin beads.

The resin and radiopaque beads may be encapsulated in a titanium tube.

The higher energy photons from the ¹⁰⁰Pd may have a higher penetration potential, in situ, than the photons of the ¹⁰³Pd.

The higher energy photons of the ¹⁰⁰Pd compared to the photons of the ¹⁰³Pd, in situ, may result in an improved anisotropy that may aid in allowing for alternative placement of the brachytherapy seeds.

According to another aspect of the invention, there is provided a method of manufacturing a brachytherapy seed that includes the steps of;
bombarding elemental silver with high energy protons produced by a cyclotron to obtain a target of ¹⁰⁰Pd and ¹⁰³Pd;
dissolving and passing the target through a column of resin beads, thereby adsorbing the target onto the resin beads;
placing at least one resin bead having adsorbed ¹⁰⁰Pd and ¹⁰³Pd into a titanium tube that is closed at one end;
inserting a radiopaque member into the titanium tube;
closing the opposite end of the titanium tube.

The ends of the titanium tube may be closed by laser welding.

### EXAMPLE OF THE INVENTION AND DESCRIPTION OF DRAWINGS

The invention will now be exemplified by the following non-limiting example and drawings;

¹⁰³Pd with a half-life of 17 days has a distinct advantage over the more commonly used ¹²⁵I (59 days half-life) in that the same effect with a much lower dose and thus less morbidity can be obtained. ¹⁰³Pd is however much more expensive to manufacture and from a cost benefit point of view is generally excluded from use in permanent implant prostate brachytherapy. ¹⁰³Pd it is the only solution considered acceptable for partial breast irradiation but the cost continues to limit the use thereof.

¹⁰³Pd is obtained by bombarding an elemental silver target with high energy protons produced by a cyclotron which also produces ¹⁰⁰Pd. It is impossible to separate isotopes (radionuclides) from each other. In order to obtain the required ¹⁰³Pd purity, the target must be left to decay until the percentage ¹⁰⁰Pd is acceptably low; however in the process up to 90% of the activity is lost.

At the time of manufacture, the mixture typically comprises 16% ¹⁰⁰Pd.

The target is dissolved and passed through a column of resin beads, sifted beforehand to obtain a desired grain size resulting in the absorption of the target onto the resin beads.

Thereafter, an open end of a 4 mm long, 0.8 mm diameter Titanium tube is laser welded closed; two Pd adsorbed resin beads are inserted into the tube; a lead or gold bead is inserted into the tube for X-ray visualization; two more resin beads are added to the tube and the opposing end of the tube are welded closed.

The inventor believes that the current invention has the advantages of dramatically reducing the cost of manufacture of a brachytherapy seed including ¹⁰⁰Pd.

The inventor also believes that the higher energy photons from the ¹⁰⁰Pd are more penetrating that that of ¹⁰³Pd and will therefore likely improve the anisotropy of the brachytherapy seed as well as allowing for a more lenient source spacing (sources can be placed further apart and small migrations will be better tolerated).

The Monte Carlo (MC) Modeling of a Brachytherapy seed is used to report on the relative absorbed dose distributions in water of a brachytherapy seed.

Benchmarked EGSnrc¹ based MC codes are used to simulate the transport of kilo-voltage photons from a model of the radioactive source located in water. In order to accomplish that the following is realized:
a) The materials constituting the source are assembled to calculate new cross-section data for electron and photon threshold energies of 1 keV. Previous data was limited to 10 keV which is, in light of the source energies, too high and is likely to introduce significant range artifacts in the dose calculation grid. Rayleigh scatter is included in said new data, and is significant to the photon energies under consideration.
b) An IDL user code is developed to enable modeling of the source, typically encapsulated. The resolution is set at 0.05 mm which is the capsule thickness. This also necessitated using smaller transport cut-off energies as described above. (Smaller resolution can lead to negative step values in the MC transport process that can produce biased results, particularly in metals).
c) A second FORTRAN code file is developed to read the model and to convert it into a suitable format for the EGSnrc MC codes to be read. This file contains the dose calculation grid, materials and densities of the materials.
d) The energy spectra files are set up as counts per bin whilst ensuring that averaging effects do not distort the energy spectra. The energy spectra that is used in the calculation is rounded off to exclude decay modes with percentage probabilities less than 2.1 percent, as it is accepted that these percentage probabilities do not have a significant impact on the absorbed dose distributions.
e) MC simulations are carried out with the following transport parameters set, keeping in mind that most of them can be safely ‘turned off’ for megavoltage photon transport:

| Transport parameter | Choice |
|---|---|
| ECUT | 0.512 MeV |
| POUT | 0.001 keV |
| Range rejection | Off |
| Boundary crossing algorithm | Exact |
| Electron step algorithm | PRESTAII |
| Electron impact ionization | On |
| Spin effects | On |
| Bound Compton scattering | On |
| Rayleigh Scattering | On |
| Atomic relaxations | On |

f) Eight simulations of one billion histories each are run, four of the simulations using energy spectrum A and four of the simulations using energy spectrum B. A FORTRAN code is utilised to sum the eight resulting dose files after the dose files for each isotope is properly weighted. Factors that are taken into account include, a) The percentage of each isotope in the combined brachytherapy source; b) the half-life of each source; and c) the fact that the brachytherapy seeds are likely to stay in the patient well after the radionuclides have decayed completely. A weighting factor to account for these factors is determined as follows:

$$w = C {\int }_{0}^{∞} {e}^{- λ t} d t = \frac{C}{λ}$$

The interpretation of the dose weighting is:

Isotope A constitutes (C=15 percent) of that for B. Its half-life (t_{1/2}) is much shorter than for isotope B (3.6 d vs. 16.99 d). The activity constants (λ=In2/t_{1/2}) have the numerical values of 0.1925 (A) and 0.0407 for B. The weight factor for Isotope A is 15/0.1925=77.922 and B is 100/0.0407=2457.00. The dose weighting factors account for the total accumulative dose from time zero.

Aforementioned weighted dose files are added and normalized at a point in water adjacent to the capsule material. Dose distributions described herein will therefore include the cumulative relative dose after an infinite time.

FIG. 1 shows the geometry of the brachytherapy seed as constructed using the IDL code. Dimensions and materials are not disclosed as this is already known. The IDL-generated model of the seed contains 5 spheres of known materials encapsulated in a metal material. This model has been used in MC simulations.

The relative dose distributions are adapted and the dose in water adjacent to the capsule wall is normalized to 1000 percent. The dose inside the capsule is capped at this value in order to derive a useful isodose data.

FIG. 2: Isodose distribution for the brachytherapy seed in the y=0 plane. The Z-axis is vertical and the X-axis horizontal (left to right). The color-bar on the right shows the activated isodose values. A 50 percent case is exemplified in this example. The source orientation matches that shown in FIG. 1.

The 600 Percent isodose line is located at about 1 mm from the capsule wall. Adjacent to the capsule the dose was normalized to 1000 percent in water. The 30 percent isodose line reaches to about 5 mm from the center of the source along the X-axis.

The view shown in FIG. 3 is in the Z=0 plane. The isodose lines through the axis can be taken as true distances. At other angles, for example, 45 degrees from these main axes, the isodose lines forms a ‘dent’ which may be considered as ‘slight’ artifacts. The isodose lines should be circular in the xy-plane due to cylindrical symmetry of the source capsule. Round-off errors in the capsule modelling causes longer effective paths for the photons traversing the capsule, causing more attenuation which are not present in the real case. The resolution is in the order of the capsule thickness, thus at 45 degrees the effective thickness is about 1.414 times thicker compared to the modeled capsule thickness on the main axis. If the source resolution is smaller (to reduce these dent effects) the MC code will encountered step length errors which will alter the isodose data significantly. This necessitates keeping the resolution grid at 0.05 mm.

FIG. 3: Isodose distribution for the brachytherapy seed in the z=0 plane. The Y-axis goes from top to bottom (X from left to right). The color-bar on the right shows the activated isodose values. The ‘dents’ in the isodose lines (prominent for the 20 and 10 percent isodose lines) are due to more attenuation in the modeled capsule thickness. The left and right parts of the dose are averaged in this plane giving rise to the symmetrical shape about the Y-axis. (The 5 percent isodose line is omitted in this Figure).

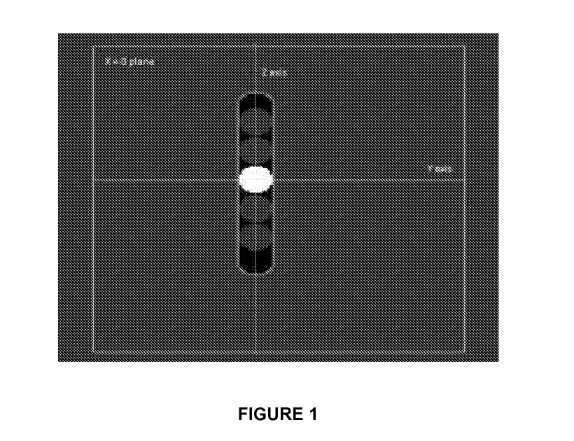

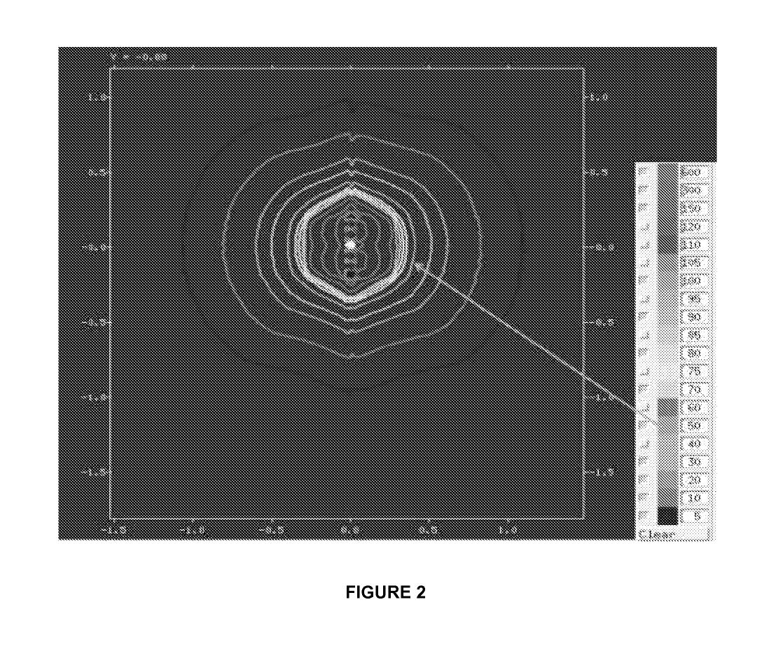

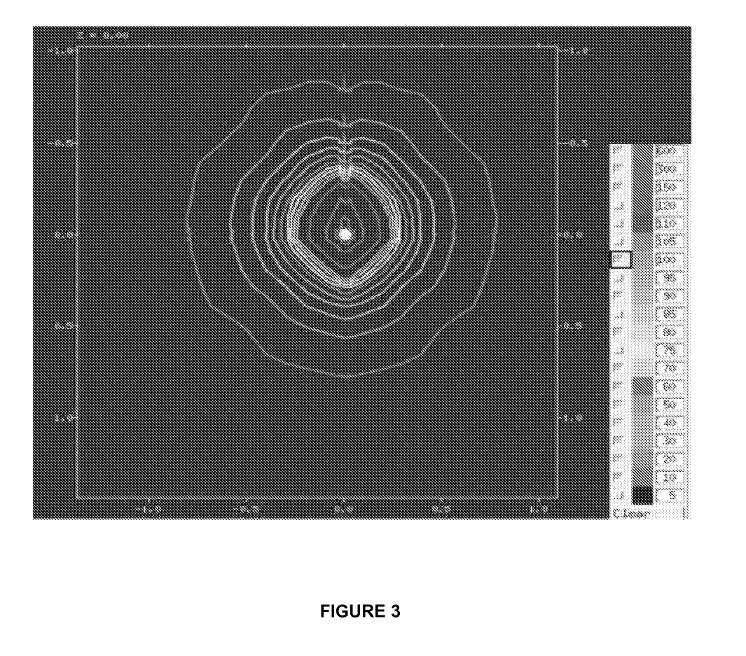

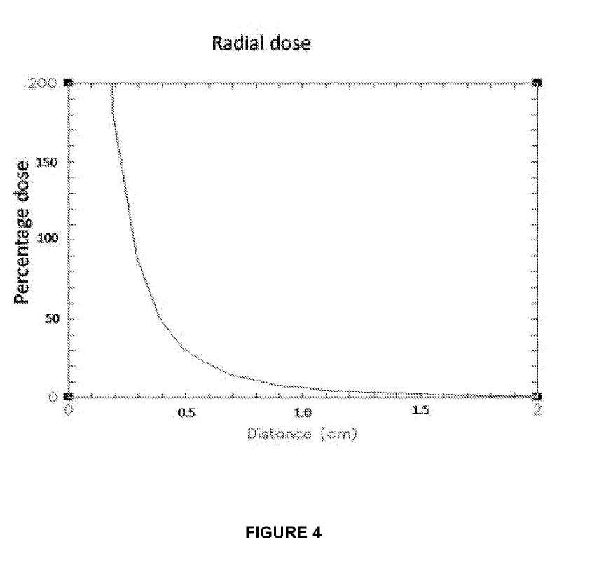

FIG. 4 shows the radial dose profile along the X axis for the data in FIG. 1. The data is normalized to 1000 percent at about 0.4 mm from the origin in water adjacent to the capsule wall location. As expected, low energy photons, which dominate the energy components in the sources effects, would be absorbed strongly due to higher interaction cross-section coefficients. The effect is a steep dose gradient falling rapidly to about 30 percent at 5 mm from the source center. Normalize the dose in water adjacent to the capsule was necessary as photo-electric absorption in the encapsulation increased the dose to about 35 times of that in water just adjacent to the capsule metal wall.

The radial dose profile is taken along the positive X-axis to avoid capsule thickness biasing effect at 45 degrees. Table 1 shows the numerical values for the radial dose profile.

FIG. 4 shows the radial dose profile normalized to 1000 percent to a point in water located adjacent to the capsule wall (0.4 mm from the zero distance position).

TABLE 1
Radial dose profile taken from the capsulation wall at 0.04 cm
to 4.1 cm

| Dist | Dose | Dist | Dose | Dist | Dose | Dist | Dose |
|---|---|---|---|---|---|---|---|
| (cm) | % | (cm) | % | (cm) | % | (cm) | % |
| 0.04 | 1000.00 | 1.09 | 4.70491 | 2.19 | 0.76853 | 3.29 | 0.240928 |
| 0.09 | 490.193 | 1.19 | 3.96427 | 2.29 | 0.409765 | 3.39 | 0.116955 |
| 0.19 | 181.845 | 1.29 | 3.4178 | 2.39 | 0.728984 | 3.49 | 0.083677 |
| 0.29 | 89.8542 | 1.39 | 2.7141 | 2.49 | 0.586933 | 3.59 | 0.204636 |
| 0.39 | 50.4976 | 1.49 | 2.22735 | 2.59 | 0.37798 | 3.69 | 0.059105 |
| 0.49 | 30.5062 | 1.59 | 2.18036 | 2.69 | 0.426131 | 3.79 | 0.059452 |
| 0.59 | 21.9893 | 1.69 | 1.52535 | 2.79 | 0.283434 | 3.89 | 0.064542 |
| 0.69 | 14.3114 | 1.79 | 1.13052 | 2.89 | 0.345896 | 3.99 | 0.138185 |
| 0.79 | 11.5428 | 1.89 | 0.900217 | 2.99 | 0.222585 | 4.09 | 0.053555 |

The water bath dimensions are 30×30×30 cm², to account for full in-phantom scatter. The voxel size of 1 mm outside the source overestimates the dose by less than 3 percent within the first 1 cm, but decreases to less than one percent thereafter. This is mainly caused by the steep dose gradient depicted in FIG. 4. The voxel resolution of the source was 0.05×0.05×0.05 mm² which reduced biased dose errors to less than one percent. Beyond the source, the phantom resolution was 1×1×1 mm². The above findings were based on the work of Taylor et al¹ where brachytherapy source dose distributions were simulated with the EGSnrc MC code and some isotope data have been compared with AAPM's TG43 recommendations.

The same transport parameters are used to simulate the brachytherapy source as recommended in the work of Taylor et al. The dose data may be slightly overestimated by 3 percent near the source. At locations beyond 1 cm this effect is well within one percent. The statistical variance within the data is below 1 percent for the outer voxels and well within this margin near the source origin. The overall uncertainty in the data is well within 3.5 percent with the overestimation of 3 percent in the dose near the source within a 1 cm boundary. Since this can be corrected for the overall uncertainty may fall below 2 percent.

### REFERENCE

¹ R. E. P Taylor and D. W. O. Rogers, “Benchmarking BrachyDose: Voxel based EGSnrc Monte Carlo calculations of TG-43 dosimetry parameters,” Med. Phys. 34, 445-457 (2007).

1. A brachytherapy seed, wherein the seed includes at least two disparate radionuclides of a chemical element, the radionuclides encapsulated to form a brachytherapy seed and wherein the combination of the disparate radionuclides of the chemical element is used to augment dosimetric parameters and radiobiological characteristics of the brachytherapy seed.

2. A brachytherapy seed as claimed in claim 1, wherein the disparate radionuclides have different half-lives, which result in different energy and decay properties.

3. A brachytherapy seed as claimed in any one of the preceding claims, wherein the dosimetric parameters of the disparate radionuclides are different, and the final dose distribution and radiobiological effectiveness of the resultant radiation being dependent on the combination of the dosimetric characteristics of the disparate radionuclides used in the brachytherapy seed.

4. A brachytherapy seed as claimed in any one of the preceding claims, wherein a ratio of the disparate radionuclides, and therefore the dosimetry of the disparate radionuclides, differs from the time of calibration to the time of use.

5. A brachytherapy seed as claimed in claim 4, wherein the ratio of the disparate radionuclides at the time of use is derived from a ratio of the disparate radionuclides at time of calibration.

6. A brachytherapy seed as claimed in any one of claims 4 to 5, wherein the ratio of the disparate radionuclides at time of use is derived from the ratio of the disparate radionuclides at time of calibration by using the published half-lives of the radionuclides and the decay equation A=A₀e^{-In(2).t/t1/2}

7. A brachytherapy seed as claimed in any one of the preceding claims, wherein the dosimetric parameters of the brachytherapy seed are determined by using mathematical modelling and wherein the disparate radionuclides are considered separately and the theoretical dose distributions are calculated and converted into desired dosimetric parameters.

8. A brachytherapy seed as claimed in claim 7, wherein the mathematical modelling is the Monte Carlo simulation, wherein the disparate radionuclides are considered separately and the theoretic dose distributions are calculated and converted into TG43 parameters¹.
¹ AAPM Radiation Therapy Task Group No. 43 (Med Phys 22(2) February 1995: 209-234 updated Med Phys 31 (3) March 2004: 633-674)

9. A brachytherapy seed as claimed in any one of the preceding claims, wherein the dosimetric parameters are determined by measuring the dosimetry of an actual seed at two or more different times, the times being long enough to show material changes in the dosimetry.

10. A brachytherapy seed as claimed in any one of the preceding claims, wherein the dosimetric parameters are determined by using a linear quadratic model to _{WO 2012/066498} e relative biological effective (RBE) of the _{PCT/IB2011/055151} [symbol] re encapsulating the disparate radionuclides.

11. A brachytherapy seed as claimed in anyone of claims 4 to 10, wherein utilising the known changes in the ratios and the measured changes in the dose distribution may assist in determining the dosimetric contributions of the disparate radionuclides.

12. A brachytherapy seed as claimed in any one of the preceding claims, wherein the disparate radionuclides are the ¹⁰⁰Pd and the ¹⁰³Pd radionuclides.

13. A brachytherapy seed as claimed in claim 12, wherein the ¹⁰⁰Pd and the ¹⁰³Pd radionuclides are obtained by bombarding a natural silver target with high energy protons produced by a cyclotron.

14. A brachytherapy seed as claimed in any one of claims 12 to 13, wherein the brachytherapy seed, at the time of manufacture, includes a range of 5-25% ¹⁰⁰Pd.

15. A brachytherapy seed as claimed in claim 14, wherein the brachytherapy seed, at the time of manufacture, includes 16% ¹⁰⁰Pd.

16. A brachytherapy seed as claimed in any one of the preceding claims, wherein the dosimetry and half-life of each disparate radionuclide is calculated separately.

17. A brachytherapy seed as claimed in any one of claims 12 to 16, wherein the ¹⁰⁰Pd dosimetry is weighted, thereby permitting the dose of the ¹⁰⁰Pb to be brought into line with the RBE of the ¹⁰³Pd before the finalisation of the dosimetry.

18. A brachytherapy seed as claimed in claim 17, wherein the weighing of the ¹⁰⁰Pd dosimetry results in a dosimetric equivalent of a pure ¹⁰³Pd seed.

19. A brachytherapy seed as claimed in any one of the preceding claims, wherein the brachytherapy seed includes a column of resin beads, for the radionuclides to absorb thereon.

20. A brachytherapy seed as claimed in any one of the preceding claims, wherein the brachytherapy seed includes a radiopaque substance for radiographic visualization of the brachytherapy seed.

21. A brachytherapy seed as claimed in claim 20, wherein the radiopaque substance is sandwiched between the resin beads.

22. A brachytherapy seed as claimed in any one of claims 19 to 21, wherein the resin beads and the radiopaque substance are encapsulated in a titanium tube.

23. A method of manufacturing a brachytherapy seed that includes the steps of;
bombarding elemental silver with high energy protons produced by a cyclotron to obtain a target of ¹⁰⁰Pd and the ¹⁰³Pd;
dissolving and passing the target through a column of resin beads, thereby adsorbing the target onto the resin beads;
placing at least one resin bead having adsorbed ¹⁰⁰Pd and the ¹⁰³Pd into a titanium tube that is closed at one end;
inserting a radiopaque member into the titanium tube;
closing the opposite end of the titanium tube

24. A method of manufacturing a brachytherapy seed as claimed in claim 23, wherein the ends of the titanium tube may be closed by laser welding.

25. A new brachytherapy seed as claimed in claim 1.

26. A brachytherapy seed substantially as herein described and illustrated.

27. A new method of manufacturing a brachytherapy seed as claimed in claim 23.

28. A new method of manufacturing a brachytherapy seed substantially as herein described and illustrated.
